(19) United States
(12) Patent Application Publication
(10) Pub. No.: US 2003/0041366 A1
(43) Pub. Date: Mar. 6, 2003

(54) Free-size cap capable of fitting a wide range of head sizes

(76) Inventor: Yum Ming Louis Ng, Kowloon (CN)

Correspondence Address: ALIX YALE & RISTAS LLP, 750 MAIN STREET, SUITE 1400, HARTFORD, CT, 06103, US

(21) Appl. No.: 10/068,377

(22) Filed: Feb. 6, 2002

Related U.S. Application Data
(63) Continuation-in-part of application No. 09/941,136, filed on Aug. 28, 2001, now abandoned.

(51) Int. Cl. A61F 9/00

(52) U.S. Cl. 2/195.1; 2/12

(57) ABSTRACT

A multi-size cap is capable of fitting head sizes of most wearers. Its crown is formed by multiple woven fabrics with high extensibility. An elastic band with high extensibility is sewn to the lower edge of the woven fabrics of the multi-size cap in some embodiments. Because the elastic band is partly exposed outside, the cap is capable of fitting wearers having different head sizes with comfort, and may be suitable for more than 95 percent of potential wearers. The cap eliminates the inconvenience of adjusting the cap and reduces the cost of manufacture. Other embodiments stitch the band to the lowermost extremity of the crown with band overlapping the crown.

### CROSS-REFERENCE TO RELATED APPLICATION

This application is a continuation-in-part of U.S. patent application Ser. No. 09/941,136 filed Aug. 28, 2001.

### BACKGROUND OF THE INVENTION

This invention relates generally to clothing and particularly to a kind of cap, especially a free-size or multi-size cap. A traditional free-size or multi-size cap has an arc-shaped open area at the back of the cap with an adjustable-length strap and fastener fitted at the two sides of the open area. In this conventional cap, the open area, strap and fastener form a size adjustment arrangement.

Another free-sized cap is shown in U.S. Pat. No. 5,715,540, in which the crown and sweatband are made from a stretchable woven fabric, that is stretchable in a latitudinal direction. Because an elastic sweatband is disposed at the inner circle of the cap, the extension of the sweatband is limited by the material of the cap-body. This cap is only capable of fitting wearers having a head size range that is a maximum of 2 inches.

Another cap, shown in Chinese Patent No. CN 00 104 381.1, has a size that can be adjusted with an adjustable-length strap by the hands of the user. The hat can then be self-fitted over a range of several inches taking advantages of the extensibility of the sweatband. The noted kinds of free-size caps either only fit a small range of head sizes or require installation of an adjustable-length strap so as to regulate the size of cap by hand. These caps rely on latitudinal extensibility of the crown and sweatband for such small adjustments. co

A cap such as described requires manual adjustment and the adjustment is not continuously adjustable. In other words, the adjustment involve a plurality of discrete steps. Accordingly, such caps do not fit well, are inconvenient, look cumbersome and are not aesthetically pleasing. In addition the addition of the adjusting structure requires complicated technology and increased production costs.

### SUMMARY OF THE INVENTION

In consideration of the above-mentioned factors, it is accordingly an object of this invention to provide a cap that adjusts to a wide range of head sizes without manual adjustment.

The cap in accordance with the present invention includes a crown that is formed by multiple woven fabrics (2 to 6 pieces) which are each stretchable in two directions. Every fabric panel has the shape of a triangle or other polygon. The latitudinal extensibility of the fabrics is larger than the longitudinal extensibility. The lower edge of the fabrics is connected with an elastic band with high extensibility. Because the extensibility of the highly elastic band is much higher than the latitudinal extensibility of the fabrics, the construction avoids the characteristic wherein the crown portion of the cap prevents full extension of the elastic band. For this purpose, the elastic band should be placed at the outer surface of the lower edge of the cap, or exposed outside as a hatband.

To obtain the above objects, the latitudinal extensibility value of fabrics should be much less than the latitudinal extensibility of the elastic band. For a highly elastic band, at least one third of the elastic band width should be exposed outside the lower edge of the cap. The latitudinal extensibility of the elastic band is preferably about 75%. The latitudinal extensibility of the fabrics that form the crown is preferably about 10%. With this combination the range of extension possibilities for the elastic band is maximized.

### BRIEF DESCRIPTION OF THE DRAWINGS

The present invention will be described in greater detail below with reference to examples and attached drawings.

FIG. 1 is a side view of a first embodiment of a cap in accordance with the invention;

FIG. 2 is a rear elevation view of the cap of FIG. 1;

FIG. 3 is a perspective view of a second embodiment of a cap in accordance with the invention;

FIG. 4 is a rear elevation view of the cap of FIG. 3;

FIG. 5 is a perspective view of a third embodiment of a cap in accordance with the invention; and

FIG. 6 is a rear elevation view, partially broken away, of the cap of FIG. 5.

### DETAILED DESCRIPTION OF THE PREFERRED EMBODIMENTS

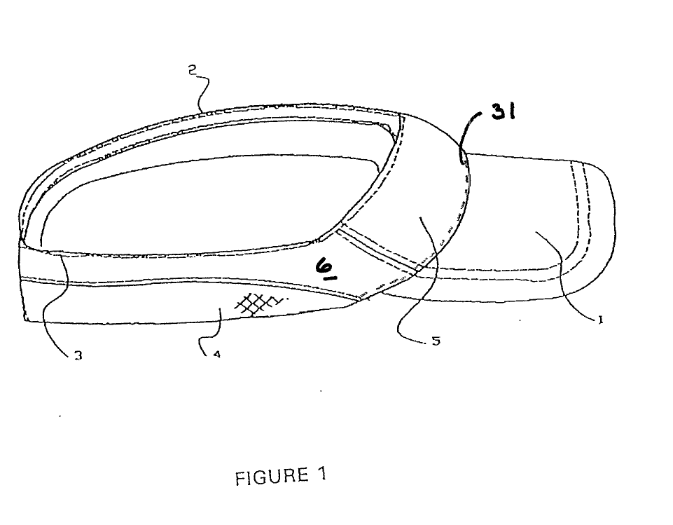

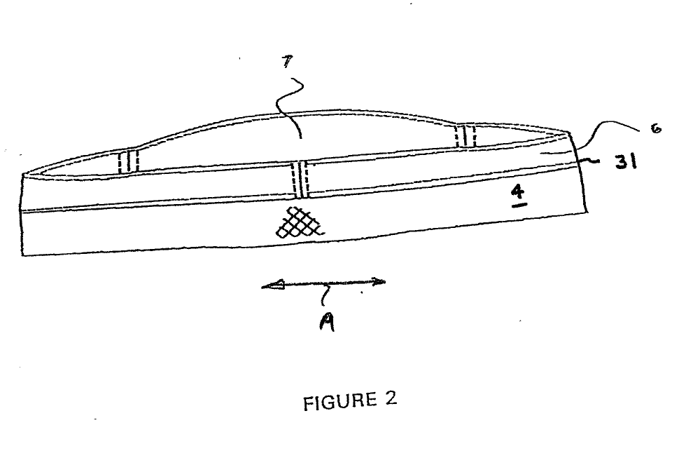

As shown in FIG. 1 and FIG. 2, the first embodiment is an open-top cap, which includes an open crown 2 and a visor 1. The open crown 2 is composed of two symmetrical side cloths 6, that each are elongated and have an axial portion that is relatively narrow at the rear and sides and an axial portion near the visor that is relatively wide. The side cloths 6 are tapered intermediate the relatively narrow and wide portions and a stretchable front fabric panel that is a generally quadrilateral panel 5. The panels 5, 6 collectively form an arc-shape (as viewed from the top and bottom). The two symmetrical side cloths 6 are sewn together at the rear of the cap and to the fabric panel 5 with a stretchable thread 3 of synthetic fiber to form the open crown 2. The lower edge of the crown 2 is also sewn to a highly stretchable rubber or elastic band 4 using a stretchable thread 3 of synthetic fiber to form the cap. The elastic band 4 extends completely around the entire extent of the cap. A single line of stitching joins the elastic band 4 to the rest of the cap. That single line of stitching 31 extends through the top edge of the elastic band 4 at the sides and rear of the cap. The line of stitching 31 extends obliquely with respect to the elastic band 4 along the tapering portions thereof. The stitching 31 proximate to the visor 1 engages the lowest edge of the elastic band 4.

The elastic band 4 is elongated and narrow, and therefore its latitudinal extensibility is a decisive parameter. The latitudinal extensibility of elastic band 4 is selected to be between 60% to 90% although about 75% is preferred. The latitudinal extensibility of the open end of the fabric panel 5 is selected to be within the range of 5% to 20% although about 10% is preferred. This range of latitudinal extensibility applies to each of the embodiments herein. The fabric panels may be knitted or woven, although woven is preferred.

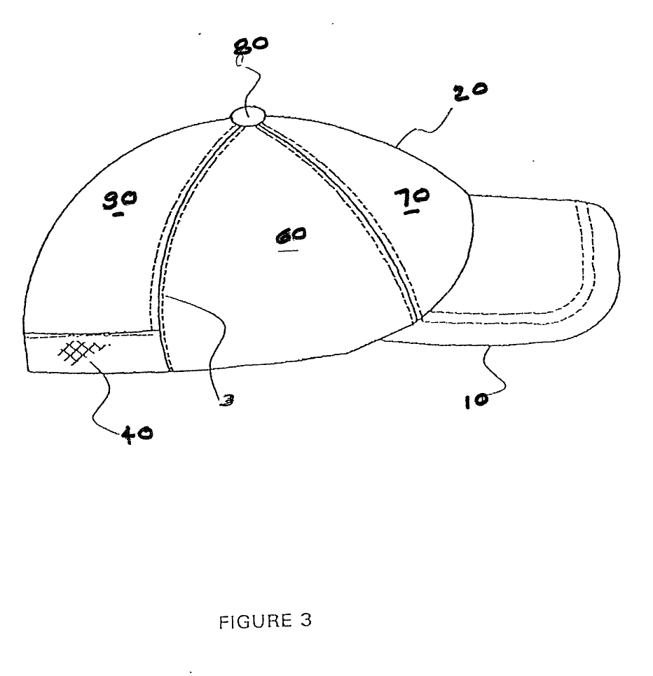

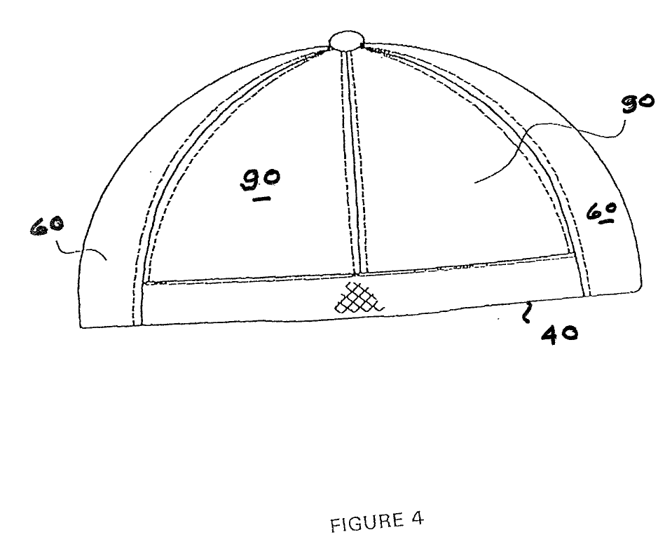

Referring now to FIGS. 3 and 4 there is shown a second embodiment. The direction of latitudinal extensibility is illustrated by the arrow A of FIG. 2. As in the first embodiment, the latitudinal extensibility of the elastic band 40 should be substantially larger than the fabric panels 60, 90 so that the range of possible sizes will be larger. When the user puts on the cap, the size will self-adjust and fit the head in comfort with uniform tension.

The band 40 preferably overlaps the lower most part of the crown 20 at the rear of the cap. The band 40 is a loop that extends inside all of the rest of the crown at the lower most part thereof.

As shown in FIG. 3 and FIG. 4, the general construction and extensibility of the fabric panels or gore fabric panels that form the crown and the elastic band 40 of the second embodiment, are the same as for the first embodiment. A primary difference is the shape of the crown 20. The crown 20 of the second embodiment is formed by six gore fabric panels or fabric panels 60, 70, 90. The seams therebetween are sewn together using stretchable thread 3 of synthetic fiber. The six gore fabric panels or fabric panels include two front cloths 70, the two side cloths 60 and two back cloths 90. The two side cloths 60 have a height greater than the two back cloths 90 by the height of the elastic band 40. As a result, when the elastic band 40 and the cloths 60, 70, 90 are sewn together using stretchable thread 3 of synthetic fiber, they each extend to the same height. Thus, the lower edge of the cap has a neat uniform lowermost extremity. A visor 10 is sewn to the lower edge of the front cloth 70 and part of the cloths 60. The top of the crown 20 is fitted with top button 80. The top button 80 is employed to facilitate finished sewing workmanship and as an adornment, which integrates these two functions into one article to provide an elegant look.

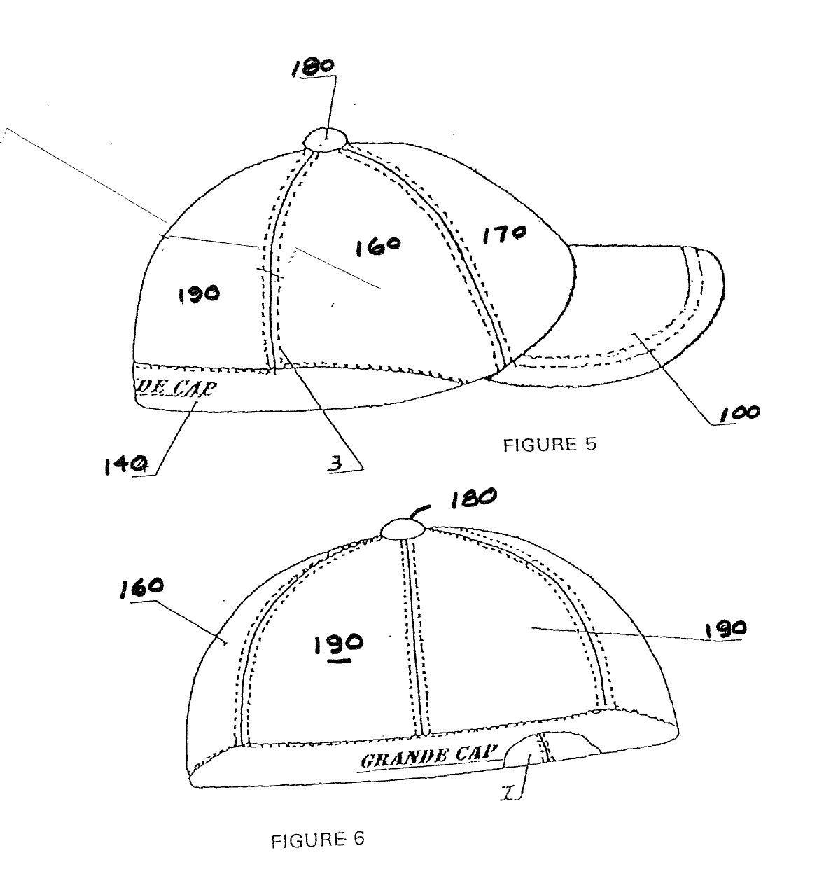

As shown in FIG. 5 and FIG. 6, the construction and extensibility of fabric panels 160, 170, 190 and elastic band 140 of the third embodiment, are similar to corresponding parts of the second embodiment. An axial portion of the band 140 that is visible in FIG. 5 extends below the cloth 160. This lateral part thereof allows adjustability. A difference is the shape of the elastic rubber band 140 that is visible. Marks and patterns are also positioned on the outer exposed portion of the band 140. The part of the elastic rubber band 40 of the second embodiment that is visible when the hat is worn has the shape of a rectangle. The visible part is an axial part of a band having a uniform width that has the ends thereof sewn together to form a continuous loop. The part of the band 40 that is visible in FIGS. 3 and 4 is generally rectangular. The shape of the elastic rubber band 140 of the third embodiment is the same, however, the part that is visible is substantially the shape of a trapezoid. None of the embodiments of the invention have a sweat band.

The fabrics and highly elastic band of this invention all possesses high extensibility. In preferred embodiments, the elongated elastic band has at a least an axial part that has a lateral part exposed below the crown. This exposure increases the range of possible extension of the band because the constraint imposed by the fabric is minimized. In this way, the cap is capable of fitting a larger number of wearers, which may exceed more than 95 percent of cap wearers, even if the crown and band do not fit exactly. In some embodiments the substantially the entire width of the band is below the crown. The manufacture of the cap in accordance with the present invention is simplified because the construction of this invention is simple. Accordingly, production costs are greatly reduced.

While preferred embodiments have been set forth for purposes of description the invention, the foregoing should not be deemed a limitation of the invention herein. For example, the open crown embodiment may be manufactured with a closed crown and the closed crown embodiments may be manufactured with an open crown. Accordingly, various modification, alternatives and adaptations may occur to one skilled in the art without departing from the spirit and scope of the present invention.

What is claimed is:

1. A multi-size cap capable of fitting multiple head sizes comprising a crown formed by multiple fabric panels, each fabric panel having a substantially polygon shape with adjacent panels being sewn together, the latitudinal extensibility of said fabric panels being larger than longitudinal extensibility, said crown having a lower edge, an elongated elastic band of high extensibility sewn to said lower edge, the latitudinal extensibility of said elastic band being larger than the latitudinal extensibility of the fabric panels, the elastic band sewn to said lower edge having an axial part thereof of which a substantial part of the entire lateral extent thereof is disposed below said crown.

2. A multi-size cap in accordance with claim 1, further including a visor and wherein the band has substantially the entire lateral extent thereof that is not adjacent to said visor disposed below the lower edge crown portion of the cap.

3. A multi-size cap in accordance with claim 1, wherein the value of latitudinal extensibility of said elastic band is between 60% and 90%.

4. A multi-size cap in accordance with claim 2, wherein the value of latitudinal extensibility of said elastic band is about 75%.

5. A multi-size cap in accordance with claim 1, wherein the latitudinal extensibility of said fabric panels is between 5% to 20%.

6. A multi-size cap in accordance with claim 1, wherein the latitudinal extensibility of said fabric panels is about 10%.

7. A multi-size cap in accordance with claim 1, wherein there are indicia on the outer surface of that portion of said elastic band which is exposed outside the lower edge crown portion of the cap.

8. A multi-size cap in accordance with claim 2, wherein there are indicia on the outer surface of that portion of said elastic band which is exposed outside the lower edge crown portion of the cap.

9. A multi-size cap in accordance with claim 8, wherein the axial extremities of the band are generally perpendicular to the direction of elongation and the axial extremities substantially abut and are sewn to fabric panels.

10. A multi-size cap in accordance with claim 9, having only a single line of stitches attaching said elastic band.

11. A multi-size cap in accordance with claim 10, wherein said single line of stitches extends along the uppermost part of said elastic band along an axial part thereof and along the lowermost part thereof along another axial part thereof.

12. A multi-size cap capable of fitting multiple head sizes comprising a crown formed by multiple fabric panels, each fabric panel having a substantially polygon shape and the latitudinal extensibility of said fabric panels being larger than longitudinal extensibility, said crown having a lower edge, an elastic band of high extensibility sewn to said lower crown portion, the latitudinal extensibility of said elastic band being larger than the latitudinal extensibility of the fabric panels, the elastic band having at least an axial part thereof disposed outside said crown, and another axial part disposed within said crown, adjacent panels being sewn together with a stretchable thread of synthetic material.

13. A multi-size cap in accordance with claim 1, wherein the band is secured to said crown by first and second lines of stitches extending along respective longitudinal edges.

14. A multi-size cap in accordance with claim 1, wherein the value of latitudinal extensibility of said elastic band is between 60% and 90%.

15. A multi-size cap in accordance with claim 12, wherein the value of latitudinal extensibility of said elastic band is about 75%.

16. A multi-size cap in accordance with claim 10, wherein the latitudinal extensibility of said fabric panels is between 5% to 20%.

17. A multi-size cap in accordance with claim 14, wherein the latitudinal extensibility of said fabric panels is about 10%.

18. A multi-size cap in accordance with claim 10, wherein there are marks and patterns on the outer surface of that portion of said elastic band which is exposed outside the lower edge crown portion of the cap.

19. A multi-size cap in accordance with claim 11, wherein there are marks and patterns on the outer surface of that portion of said elastic band which is exposed outside the lower edge crown portion of the cap.

20. A multi-size cap in accordance with claim 17, wherein the axial extremities of the band are generally perpendicular to the direction of elongation and the axial extremities substantially abut and are sewn to fabric panels.

21. A multi-size cap wherein the part of said band visible outside of said cap is generally rectangular.

22. A multi-size cap wherein the part of said band visible outside of said cap is generally trapezoidal.

23. A multi-size cap in accordance with claim 1 that does not consist of a sweatband.

24. A multi-size cap in accordance with claim 12 that does not consist of a sweatband.
